(19) United States
(12) Patent Application Publication
(10) Pub. No.: US 2026/0034035 A1
(43) Pub. Date: Feb. 5, 2026

(54) ORAL COMPOSITION FOR TOOTH WHITENING

(71) Applicant: AE KYUNG INDUSTRIAL CO., LTD, Seoul (KR)

(72) Inventors: Yun Ju OH, Daejeon (KR); Kyong Hoon SHIN, Daejeon (KR)

(21) Appl. No.: 19/276,906

(22) Filed: Jul. 22, 2025

(30) Foreign Application Priority Data
Jul. 30, 2024 (KR) 10-2024-0100854

(51) Int. Cl. A61K 8/22; A61K 8/24; A61K 8/25; A61Q 11/00

(52) U.S. Cl. CPC A61K 8/22; A61K 8/24; A61K 8/25; A61Q 11/00

(57) ABSTRACT

The present disclosure relates to an oral composition for tooth whitening, which contains a peroxide, a polyvalent phosphate, a divalent metal phosphate, and water.

### CROSS-REFERENCE TO RELATED APPLICATIONS

The present application claims priority to Korean Patent Application No. 10-2024-0100854 filed on Jul. 30, 2024, the disclosure of which is incorporated herein by reference.

### STATEMENT RE: FEDERALLY SPONSORED RESEARCH/DEVELOPMENT

Not Applicable

### BACKGROUND

### 1. Field of the Invention

The present disclosure relates to an oral composition for tooth whitening.

### 2. Description of the Related Art

Factors for tooth discoloration, which are currently known, can be divided into endogenous tooth discoloration caused by nerve damage inside the tooth, aging, or overdose of antibiotics such as tetracycline, and exogenous tooth discoloration caused by food residues deposited on the tooth surface or nicotine and tar components in cigarettes, coffee, and black tea.

In order to prevent discoloration of the tooth that has been discolored due to such various causes, a technique related to an oral composition for tooth whitening has long been known publicly, and most toothpastes for tooth whitening that are currently on the market contain a peroxide, such as hydrogen peroxide that releases active oxygen.

It is already well known that the above-described peroxide is effective in tooth whitening, however, it is not simple to formulate the peroxide into a toothpaste product due to the reactivity of commonly used toothpaste components, in particular, water, an abrasive agent, and a peroxide. In addition, there is a limitation in that storage stability is deteriorated due to this reactivity.

In addition, although a peroxide is stable when the pH is low, oxidation is more prevalent than whitening, and whitening action is known to be more prevalent than the oxidation action when the pH is high, however, the peroxide is quickly decomposed into water and hydrogen, which is disadvantageous in that storage for a long period of time is difficult. As a result, in a case of being stored for a long period of time, the toothpaste container may swell or burst, and the contents may leak out, and as a result, enough peroxide may not be left to effectively clean and whiten teeth. In addition, pH decreases during long-term storage, and when the pH decreases, it may adversely affect the erosion of enamel. In the case of enamel, it is generally known that erosion occurs at a pH of 5 or less.

As a result, in a case of an oral composition containing peroxide in the related art, the initial content and pH of the peroxide depend greatly on the storage period, and storage before sale or storage by a user may result in unacceptable levels of the decomposition of the peroxide and the loss of whitening efficacy, and limitations have occurred due to pH degradation.

Therefore, in general, a composition for tooth whitening has been developed to have a pH at a level of 4 to 5 for the stability of hydrogen peroxide or has been developed as a formulation having a pH of 5 or more and containing almost no moisture. This is because the decomposition of the peroxide may be promoted when the pH is 5 or more and an excessive amount of moisture is contained, which may result in the loss of whitening efficacy in a case of being stored for a long period of time. Accordingly, conventionally, efforts have been made to improve stability of peroxide by reducing or eliminating water content at a high pH of 5 or more. However, in this case, limitations such as an increase in manufacturing costs and a decrease in feeling of use have occurred. As a result, a composition for tooth whitening, which contains an excessive amount of moisture so that, in particular, it is excellent in terms of cost competitiveness and feeling of use while ensuring the stability of hydrogen peroxide at a pH of 5.5 or more, thereby reducing tooth erosion, is desired.

Documents of Related Art: Korean Patent Registration No. 10-0149395.

### BRIEF SUMMARY

The present disclosure provides an oral composition for tooth whitening, which stabilizes a peroxide at a high pH and, at the same time, increases the content of water, thereby improving the feeling of use while lowering the manufacturing cost.

The object to be achieved by the present disclosure is not limited to the object mentioned above, and the other objects not mentioned will be clearly understood by a person skilled in the art from the description below.

Embodiments of the present disclosure for achieving such objects as described above provide an oral composition for tooth whitening, which contains a peroxide, a polyvalent phosphate, a divalent metal phosphate, and water, and may have a pH of 5.5 or more.

The peroxide may be one or more selected from the group consisting of substances that are capable of releasing hydrogen peroxide, and the substances that are capable of releasing hydrogen peroxide may include hydrogen peroxide, carbamide peroxide, calcium peroxide, sodium percarbonate, and sodium perborate.

The polyvalent phosphate may be one or more selected from the group consisting of polyvalent phosphates that have a linear form, cyclic form, or branched form, and the polyvalent phosphate having a linear form, cyclic form, or branched form may include sodium metaphosphate, sodium pyrophosphate, sodium polyphosphate, potassium pyrophosphate, acidic sodium pyrophosphate, acidic sodium metapolyphosphate, and acidic sodium polyphosphate.

The divalent metal phosphate may be one or more selected from the group consisting of calcium, magnesium, copper, and a phosphate thereof.

Based on a total amount of the oral composition for tooth whitening, a content of the peroxide may be 0.01 wt % to 10 wt %, a content of the polyvalent phosphate may be 0.01 wt % to 5 wt %, a content of the divalent metal phosphate may be 0.01 wt % to 2 wt %, and a content of the water may be 5 wt % to 55 wt %.

The oral composition for tooth whitening may further contain one or more selected from the group consisting of a binding agent and a solubilizing agent.

The binding agent may include one or more selected from the group consisting of substances that are capable of forming a hydrogen bond with hydrogen peroxide, and the substances that are capable of forming a hydrogen bond with the hydrogen peroxide may include polyvinyl pyrrolidone, polyacrylamide, a polyvinyl pyrrolidone-vinyl acetate copolymer, and poly(2-ethyl-2-oxazoline).

The solubilizing agent is a surfactant having an HLB of 14 to 23, and the surfactant having an HLB of 14 to 23 may be one or more selected from the group consisting of a poloxamer 407, a poloxamer 184, a polysorbate 60, a polysorbate 80, a polysorbate 20, a polyoxyethylene hydrogenated castor oil-40 (HCO 40), and a polyoxyethylene hydrogenated castor oil-60 (HCO 60).

The oral composition for tooth whitening may further contain one or more additives selected from the group consisting of an abrasive agent, a viscosity adjusting agent, a pH adjusting agent, a wetting agent, a foaming agent, a sweetening agent, and a flavoring agent.

The oral composition for tooth whitening according to the present disclosure improves the stability of hydrogen peroxide at a high pH, prevents pH and hydrogen peroxide from decreasing even during long-term storage, and maintains a tooth whitening effect while maintaining a pH higher than the pH for tooth erosion pH, thereby being capable of preventing tooth damage due to pH. In addition, since the content of water may be maintained high, it is possible to realize an excellent feeling of use together with economical advantages in terms of cost.

The effects of the present disclosure are not limited to the effects mentioned above, and the other effects not mentioned will be clearly understood by a person skilled in the art from the description below.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain principles of the present disclosure.

FIG. 1 is a photographic image of a test piece that is used in evaluating whitening efficacy; and

FIG. 2 is a graph showing the results of color difference measurement in the evaluation of whitening efficacy.

### DETAILED DESCRIPTION

The advantages and features of the present disclosure and the method for achieving them will be obvious with reference to the exemplary embodiments described in detail below. However, the present disclosure is not limited to the embodiments disclosed below but will be embodied in a variety of different forms. Rather, the present exemplary embodiments are provided so that the present disclosure will be thorough and complete, and will fully convey the scope of the present disclosure to a person skilled in the art to which the present disclosure belongs, and thus the present disclosure is defined only by the scope of the claims.

The terms used in the present specification are used for describing the embodiments and are not intended to limit the present disclosure. In the present specification, the singular form also includes the plural form unless particularly mentioned otherwise in the text. The terms “comprises” and/or “comprising”, which is used in the specification, does not exclude the presence or addition of one or more other constituent components other than the mentioned constituent element. Throughout the specification, “and/or” includes each and all combinations of one or more of the mentioned constituent elements. Although “first”, “second”, and the like are used to describe various constituent elements, it is needless to say that these constituent elements are not limited by these terms. These terms are used only to distinguish one constituent element from another. Therefore, it is needless to say that the first constituent element which will be described below may be the second constituent element within the technical idea of the present disclosure.

Unless defined otherwise, all terms (including technical and scientific terms) used in the present specification may be used to have meanings that are commonly understood by a person skilled in the art to which the present disclosure belongs. In addition, terms defined in commonly used dictionaries should not be construed ideally or excessively unless clearly and specifically defined.

Hereinafter, the present disclosure will be described in detail. The present disclosure provides an oral composition for tooth whitening, which contains a peroxide, a polyvalent phosphate, a divalent metal phosphate, and water.

In an exemplary embodiment, the pH of the oral composition for tooth whitening may be 5.5 or more, 5.5 to 8.5, 5.5 to 7.1, or 5.5 to 6.8. When the oral composition for tooth whitening has a high pH within the range described above, the tooth whitening effect may be excellent.

In an exemplary embodiment, the peroxide may be one or more selected from the group consisting of substances that are capable of releasing hydrogen peroxide. Examples of the substances that are capable of releasing hydrogen peroxide include hydrogen peroxide, carbamide peroxide, calcium peroxide, sodium percarbonate, and sodium perborate. As a specific example, the peroxide may be hydrogen peroxide.

It is well known that the peroxide is a component that releases active oxygen and has an excellent effect in tooth whitening, however, the peroxide has a limitation in that the compatibility with commonly used raw materials of the toothpaste is poor, and the storage stability decreases due to high reactivity. In addition, although a peroxide is stable when the pH is low, oxidation is more prevalent than whitening, and whitening action is known to be more prevalent than the oxidation action when the pH is high, however, the peroxide is quickly decomposed into water and hydrogen, which is disadvantageous in that storage for a long period of time is difficult. As a result, in a case of being stored for a long period of time, the toothpaste container may swell or burst, and the contents may leak out, and as a result, enough peroxide may not be left to effectively clean and whiten teeth. In addition, pH decreases during long-term storage, and when the pH decreases, it may adversely affect the erosion of enamel. As a result, in a case of an oral composition containing a peroxide in the related art, the initial content and pH of the peroxide depend greatly on the storage period, and storage before sale or storage by a user may result in unacceptable levels of the decomposition of the peroxide and the loss of whitening efficacy, and limitations have occurred due to pH degradation. In addition, in the related art, efforts have been made to improve the stability of the peroxide by reducing the content of water or not containing water at a high pH, however, in this case, limitations such as an increase in manufacturing cost and a decrease in the feeling of use occurred.

Accordingly, the present disclosure aims to provide an oral composition for tooth whitening that may improve the stability of hydrogen peroxide at a high pH, prevent pH and hydrogen peroxide from decreasing even during long-term storage, lower manufacturing costs, and achieve an excellent feeling of use.

In an exemplary embodiment, the content of the peroxide may be 0.01 wt % to 10 wt %, 0.1 wt % to 7 wt %, 0.5 wt % to 5 wt %, 0.5 wt % to 3 wt %, or 0.5 wt % to 1.5 wt %, based on the total amount of the oral composition for tooth whitening. When the peroxide is contained within the range described above, a whitening effect may be achieved.

In an exemplary embodiment, the polyvalent phosphate may be one or more selected from the group consisting of polyvalent phosphates that have a linear form, cyclic form, or branched form. Examples of the polyvalent phosphate having a linear form, cyclic form, or branched form include sodium metaphosphate, sodium pyrophosphate, sodium polyphosphate, potassium pyrophosphate, acidic sodium pyrophosphate, acidic sodium metapolyphosphate, and acidic sodium polyphosphate. As a specific example, the polyvalent phosphate may be sodium metaphosphate.

The content of the polyvalent phosphate may be 0.01 wt % to 5 wt %, 0.01 wt % to 3 wt %, 0.3 wt % to 3 wt %, or 0.3 wt % to 1.5 wt %, based on the total amount of the oral composition for tooth whitening. When the polyvalent phosphate is contained within the range described above, the peroxide stability and the pH stability may be improved.

In an exemplary embodiment, the divalent metal phosphate may be one or more selected from the group consisting of calcium, magnesium, copper, and a phosphate thereof. For example, the divalent metal phosphate may be one or more selected from the group consisting of hydroxyapatite, calcium phosphate, tricalcium phosphate, calcium dihydrogen phosphate, calcium diphosphate, calcium pyrophosphate, acidic calcium pyrophosphate, trimagnesium phosphate, dimagnesium phosphate, monomagnesium phosphate, and copper pyrophosphate. As a specific example, the divalent metal phosphate may be a phosphate containing calcium, which is one or more selected from the group consisting of hydroxyapatite, calcium phosphate, tricalcium phosphate, and calcium pyrophosphate.

The content of the divalent metal phosphate may be 0.01 wt % to 2 wt %, 0.01 wt % to 1 wt %, 0.01 wt % to 0.8 wt %, 0.05 wt % to 0.5 wt %, 0.075 wt % to 0.5 wt %, or 0.075 wt % to 0.3 wt %, based on the total amount of the oral composition for tooth whitening. When the divalent metal phosphate is contained within the range described above, the stability of hydrogen peroxide and the pH stability may be improved.

In an exemplary embodiment, the water described above, for example, purified water may be contained. The content of the water may be 5 wt % to 55 wt %, 10 wt % to 52 wt %, 35 wt % to 52 wt %, or 35 wt % to 45 wt %, based on the total amount of the oral composition for tooth whitening. When water is contained within the range described above, the stability of the peroxide may be improved.

In an exemplary embodiment, the oral composition for tooth whitening may further contain a binding agent. As the binding agent, a polymer binding agent that is one or more selected from the group consisting of substances that are capable of forming a hydrogen bond with hydrogen peroxide may be used. Examples of the polymer binding agent include polyvinyl pyrrolidone, polyacrylamide, a polyvinyl pyrrolidone-vinyl acetate copolymer, and poly(2-ethyl-2-oxazoline). The binding agent functions to trap a peroxide and may improve the stability of the peroxide.

As a specific example, the binding agent may include polyvinyl pyrrolidone (PVP). As the polyvinyl pyrrolidone, polyvinyl pyrrolidone having a linear or crosslinked form may be used without limitation. The polyvinyl pyrrolidone is a polymer that has a small effect on a reaction between a co-precipitate and a peroxide, may effectively achieve the promotion of decomposition of organic substances, and may not interfere with the release and movement of radicals to the tooth when an oral composition for tooth whitening containing a peroxide is applied.

In an exemplary embodiment, the content of the binding agent may be 0.01 wt % to 10 wt %, 0.1 wt % to 7 wt %, 0.5 wt % to 5 wt %, or 0.5 wt % to 3 wt %, based on the total amount of the oral composition for tooth whitening. When the binding agent is contained within the range described above, the stability of the peroxide in the formulation may be improved.

In an exemplary embodiment, the weight ratio of the binding agent to the peroxide may be more than 0.27, 0.35 or more, 0.53 or more, 0.53 to 5, or 0.53 to 3. When the weight ratio of the binding agent to the peroxide is adjusted within the range described above, the stability of the peroxide may be improved.

In an exemplary embodiment, the oral composition for tooth whitening may further contain a solubilizing agent. The solubilizing agent may be, for example, a surfactant including HLB 14 to HLB 23.

The surfactant having an HLB of 14 to 23 may be, for example, one or more selected from the group consisting of a poloxamer 407, a poloxamer 184, a polysorbate 60, a polysorbate 80, a polysorbate 20, a polyoxyethylene hydrogenated castor oil-40 (HCO 40), and a polyoxyethylene hydrogenated castor oil-60 (HCO 60). As a specific example, the surfactant may be a poloxamer-based surfactant. The surfactant functions to trap a peroxide with a binding agent and to additionally trap them in the formulation, thereby being capable of improving the stability of the peroxide.

The content of the surfactant may be 0.01 wt % to 5 wt %, 0.01 wt % to 3 wt %, and 1 wt % to 2 wt %, based on the total amount of the oral composition for tooth whitening. When the solubilizing agent is contained within the range described above, the stability of the peroxide may be improved.

In an exemplary embodiment, the oral composition for tooth whitening may further contain one or more additives selected from the group consisting of an abrasive agent, a viscosity adjusting agent, a pH adjusting agent, a wetting agent, a foaming agent, a sweetening agent, and a flavoring agent. Each of the abrasive agent, the viscosity adjusting agent, the pH adjusting agent, the wetting agent, the foaming agent, the sweetening agent, and the flavoring agent is not particularly limited as long as it may be contained in a general oral composition. In addition, other additives that may be contained in a general oral composition may be further contained.

The abrasive agent may include, for example, one or more selected from the group consisting of colloidal silicon dioxide, hard silicic acid anhydride, silicon dioxide, aluminum hydroxide, and insoluble sodium metaphosphate. The content of the abrasive agent is not particularly limited, however, it may be, for example, 1 wt % to 15 wt %, 1 wt % to 10 wt %, 3 wt % to 10 wt %, or 6 wt % to 8 wt %, based on the total amount of the oral composition for tooth whitening.

The viscosity adjusting agent may be one or more selected from the group consisting of polyvinyl alcohol, polyoxyethylene polyoxypropylene glycol, alginate, hydroxypropylmethyl cellulose, hydroxyethyl cellulose, methyl cellulose, carboxymethyl cellulose, xanthan gum, acrylate copolymer, and polyvinyl pyrrolidone-vinyl acetate copolymer. The content of the viscosity adjusting agent is not particularly limited, however, it may be, for example, 0.1 wt % to 5 wt %, 0.1 wt % to 3 wt %, or 0.5 wt % to 1.5 wt %, based on the total amount of the oral composition for tooth whitening.

The pH adjusting agent may be one or more selected from the group consisting of phosphoric acid, citric acid, and a salt thereof. For example, the pH adjusting agent may be one or more selected from the group consisting of phosphoric acid, sodium diphosphate, sodium monophosphate, sodium polyphosphate, sodium pyrophosphate, acidic sodium pyrophosphate, sodium triphosphate, citric acid, and sodium citrate. The content of the pH adjusting agent is not particularly limited, however, it may be, for example, 0.01 wt % to 1 wt %, 0.05 wt % to 1 wt %, 0.05 wt % to 0.5 wt %, or 0.05 wt % to 0.3 wt %, based on the total amount of the oral composition for tooth whitening.

The wetting agent may include, for example, one or more selected from the group consisting of polyethylene glycol, sorbitol, glycerin, dipropylene glycol, and propylene glycol. The content of the wetting agent is not particularly limited, however, it may be, for example, 10 wt % to 70 wt %, 10 wt % to 55 wt %, 30 wt % to 60 wt %, or 37 wt % to 50 wt %, based on the total amount of the oral composition for tooth whitening.

The wetting agent may include a first wetting agent including sorbitol and a second wetting agent including glycerin, and the first wetting agent and the second wetting agent may be contained such that a weight ratio of the first wetting agent to the second wetting agent is 1:1.5 to 1:5, 1:2 to 1:4, or 1:2 to 1:3. When the first wetting agent and the second wetting agent are mixed at a weight ratio within the range described above, the stability of hydrogen peroxide and the appearance stability may be improved while maintaining the pH of the composition at 5.5 or more.

The foaming agent may include, for example, one or more selected from the group consisting of anionic surfactants such as sodium lauryl sulfate and betaines. The content of the foaming agent is not particularly limited, however, it may be, for example, 0.1 wt % to 10 wt %, 0.1 wt % to 7 wt %, 0.1 wt % to 5 wt %, or 0.5 wt % to 3 wt %, based on the total amount of the oral composition for tooth whitening.

The sweetening agent may include, for example, one or more selected from the group consisting of saccharin, saccharin sodium, enzymatically processed stevia, sucralose, acesulfame potassium, aspartame, and xylitol. The content of the sweetening agent is not particularly limited, however, it may be, for example, 0.01 wt % to 5 wt %, 0.1 wt % to 3 wt %, or 0.01 wt % to 1 wt %, based on the total amount of the oral composition for tooth whitening.

The kind and content of the flavoring agent are not particularly limited, however, the content thereof may be, for example, 0.01 wt % to 5 wt %, 0.1 wt % to 3 wt %, or 0.5 wt % to 1.5 wt %, based on the total amount of the oral composition for tooth whitening.

The sum of the contents of the sweetening agent and the flavoring agent is not particularly limited, however, it may be 0.1 wt % to 10 wt % based on the total amount of the oral composition for tooth whitening.

Hereinafter, specific examples of the present disclosure are presented. However, the examples described below are merely for exemplarily demonstrating or describing the present disclosure in detail, and thus the present disclosure should not be limited thereto. In addition, the content that is not described here is technically inferred sufficiently by a person skilled in this technical field, and thus the description thereof is omitted.

### PREPARATION OF EXAMPLES AND COMPARATIVE EXAMPLES

### Examples 1 to 10 and Comparative Examples 1 to 3

With respect to the total amount (100 wt %) of the oral composition, calcium phosphate, sodium hexametaphosphate, hydrogen peroxide (H2O2), and purified water were adjusted as shown in Table 1 below, and, in each of examples and comparative examples, a binding agent, a solubilizing agent, a pH adjusting agent, an abrasive agent, a wetting agent, a viscosity adjusting agent, a foaming agent, a sweetening agent, and a flavoring agent were additionally mixed to prepare an oral composition for tooth whitening (unit: wt % with respect to the total weight of the composition).

The pH was measured according to the following standards.

### [Evaluation Standards]—pH

Measurement method: A diluent (10% of each test group+90% of purified water) is subjected to a measurement of pH.
Target: 5.5 or more

TABLE 1

| | | | | | | | | | | | Compar- | Compar- | Compar- |
|---|---|---|---|---|---|---|---|---|---|---|---|---|---|
| | | | | | | | | | | | ative | ative | ative |
| | Exam- | Exam- | Exam- | Exam- | Exam- | Exam- | Exam- | Exam- | Exam- | Exam- | Exam- | Exam- | Exam- |
| | ple 1 | ple 2 | ple 3 | ple 4 | ple 5 | ple 6 | ple 7 | ple 8 | ple 9 | ple 10 | ple 1 | ple 2 | ple 3 |
| Colloidal | 7 | 7 | 7 | 7 | 7 | 7 | 7 | 7 | 7 | 7 | 7 | 7 | 7 |
| silicon dioxide | | | | | | | | | | | | | |
| Hydrogen | 0.75 | 0.75 | 0.75 | 0.75 | 0.75 | 0.75 | 0.75 | 2.8 | 3.0 | 0.75 | 0.75 | 0.75 | 0.75 |
| peroxide | | | | | | | | | | | | | |
| Sorbitol | 10 | 10 | 10 | 10 | 10 | 10 | 10 | 10 | 10 | 10 | 10 | 10 | 10 |
| Glycerin | 30 | 30 | 30 | 30 | 20 | 30 | 30 | 30 | 30 | 10 | 30 | 30 | 30 |
| PVP | 2 | 2 | 2 | 2 | 2 | 2 | 1 | 2 | 2 | 2 | 2 | 2 | 2 |
| Phosphoric acid | 0.1 | 0.1 | 0.1 | 0.1 | 0.1 | 0.1 | 0.1 | 0.1 | 0.1 | 0.1 | 0.1 | 0.1 | 0.1 |
| Saccharin | 0.35 | 0.35 | 0.35 | 0.35 | 0.35 | 0.35 | 0.35 | 0.35 | 0.35 | 0.35 | 0.35 | 0.35 | 0.35 |
| sodium | | | | | | | | | | | | | |
| Poloxamer 407 | 2 | 2 | 2 | 2 | 2 | 2 | 2 | 2 | 2 | 2 | 2 | 2 | 2 |
| Xanthan gum | 0.5 | 0.5 | 0.5 | 0.5 | 0.5 | 0.5 | 0.5 | 0.5 | 0.5 | 0.5 | 0.5 | 0.5 | 0.5 |
| Sodium lauryl | 3 | 3 | 3 | 3 | 3 | 3 | 3 | 3 | 3 | 3 | 3 | 3 | 3 |
| sulfate | | | | | | | | | | | | | |
| Calcium | 0.1 | 0.3 | 0.5 | 0.075 | 0.5 | 1 | 0.1 | 0.5 | 0.5 | 0.5 | 0.5 | 0 | 0 |
| phosphate | | | | | | | | | | | | | |
| Sodium | 1 | 1 | 1.5 | 0.3 | 1.5 | 3 | 1 | 1.5 | 1.5 | 1.5 | 0 | 2 | 0 |
| metaphosphate | | | | | | | | | | | | | |
| Flavoring agent | 1 | 1 | 1 | 1 | 1 | 1 | 1 | 1 | 1 | 1 | 1 | 1 | 1 |
| Purified water | 42.2 | 42 | 41.3 | 42.925 | 51.3 | 39.3 | 43.2 | 39.25 | 39.05 | 61.3 | 42.8 | 41.3 | 43.3 |
| pH | 6.5 | 6.5 | 6.8 | 6.4 | 6.8 | 6.8 | 6.5 | 6.0 | 6.0 | 6.8 | 6.8 | 6.5 | 4.5 |

### EXPERIMENTAL EXAMPLE

### Experimental Example 1: Evaluation of Hydrogen Peroxide Stability and Appearance Stability

For the compositions of examples and comparative examples prepared above, hydrogen peroxide stability and appearance stability were evaluated according to the following method.

### [Evaluation Standards]—Hydrogen Peroxide Stability

Measurement method: After storage under acceleration conditions that make it possible to obtain results similar to those in the elapse of 3 years at ordinary temperature, the content of hydrogen peroxide is measured using a potentiometric titration method.
X: Reduction amount of hydrogen peroxide: more than 15%
∘: Reduction amount of hydrogen peroxide: 15% or less
Target: Equal to or higher than ∘

### [Evaluation Standards]—Appearance Stability

Verification method: The level of internal bubbles is evaluated after storing a tube filled with an appropriate amount of a test group, under high temperature conditions for a certain period of time.
X: Tube expansion is severe, and a lot of air bubbles are present in a case of being observed with the naked eye.
∘: Tube expansion is not severe, and large air bubbles are not present in a case of being observed with the naked eye.
Target: Equal to or higher than ∘
The evaluation results are as shown in Table 2 below.

TABLE 2

| | | | | | | | | | | | Compar- | Compar- | Compar- |
|---|---|---|---|---|---|---|---|---|---|---|---|---|---|
| | | | | | | | | | | | ative | ative | ative |
| | Exam- | Exam- | Exam- | Exam- | Exam- | Exam- | Exam- | Exam- | Exam- | Exam- | Exam- | Exam- | Exam- |
| | ple 1 | ple 2 | ple 3 | ple 4 | ple 5 | ple 6 | ple 7 | ple 8 | ple 9 | ple 10 | ple 1 | ple 2 | ple 3 |
| Hydrogen | ◯ | ◯ | ◯ | ◯ | ◯ | ◯ | ◯ | ◯ | ◯ | X | ◯ | X | ◯ |
| peroxide | | | | | | | | | | | | | |
| stability | | | | | | | | | | | | | |
| (reduced | | | | | | | | | | | | | |
| amount) | | | | | | | | | | | | | |
| Appearance | ◯ | ◯ | ◯ | ◯ | ◯ | ◯ | ◯ | ◯ | ◯ | X | X | ◯ | ◯ |
| stability | | | | | | | | | | | | | |
| (phenomenon | | | | | | | | | | | | | |
| of tube | | | | | | | | | | | | | |
| expansion) | | | | | | | | | | | | | |

With reference to Table 2, it could be confirmed that the pH is 5.5 or more, and the hydrogen peroxide stability and the appearance stability are excellent when an oral composition for tooth whitening was formed according to the composition according to the present disclosure. In the case of Example 10, it could be confirmed that a composition having low hydrogen peroxide stability and low appearance stability has been formed due to a high content of purified water.

On the other hand, in the case of Comparative Example 1, it could be confirmed that a composition having low appearance stability has been formed since a polyvalent phosphate is not contained, and in the case of Comparative Example 2, it could be confirmed that a composition having low hydrogen peroxide stability has been formed since a divalent metal phosphate is not contained. In the case of Comparative Example 3, the stability of the formed composition was favorable due to a low pH although a polyvalent phosphate and a divalent metal phosphate are not contained, however, the pH turned out to be less than 5.5.

### Experimental Example 2: Evaluation of Stability of Hydrogen Peroxide Depending on Weight Ratio of Polyvinyl Pyrrolidone/Hydrogen Peroxide

An oral composition for tooth whitening was prepared as in Example 1, and then the reduction amount of hydrogen peroxide was evaluated under high temperature conditions according to the following standards while adjusting the weight ratio of polyvinyl pyrrolidone (PVP)/hydrogen peroxide (H₂O₂) to 0.27 to 2.66, and the results are shown in Table 3.

### [Evaluation Standards]

X: More than 15%
Δ: More than 10% and 15% or less
∘: 10% or less
(Target: Equal to or higher than Δ)

TABLE 3

| | Example 11 | Example 12 | Example 13 | Example 14 | Example 15 | Example 16 | Example 17 |
|---|---|---|---|---|---|---|---|
| PVP Content | 0.2 | 0.4 | 0.5 | 0.6 | 0.8 | 1 | 2 |
| (wt %) | | | | | | | |
| H₂O₂ Content | 0.75 | 0.75 | 0.75 | 0.75 | 0.75 | 0.75 | 0.75 |
| (wt %) | | | | | | | |
| PVP/H₂O₂ | 0.27 | 0.53 | 0.67 | 0.8 | 1.07 | 1.33 | 2.66 |
| weight ratio | | | | | | | |
| H₂O₂ | X | Δ | ◯ | ◯ | ◯ | ◯ | ◯ |
| reduced | | | | | | | |
| amount | | | | | | | |

With reference to Table 3, it could be confirmed that when the weight ratio of polyvinyl pyrrolidone/hydrogen peroxide is 0.27, the reduction amount of hydrogen peroxide increases and the stability of the hydrogen peroxide deteriorates, and it could be confirmed that the evaluation result of hydrogen peroxide is equal to or higher than Δ when the weight ratio is more than 0.27, and satisfies the target physical properties.

### Experimental Example 3: Evaluation of Whitening Efficacy of Oral Composition for Tooth Whitening (In Vitro)

Whitening efficacy was evaluated for each of the oral compositions for tooth whitening according to Examples 1 and 2 and the toothpaste of Reference Example 1.

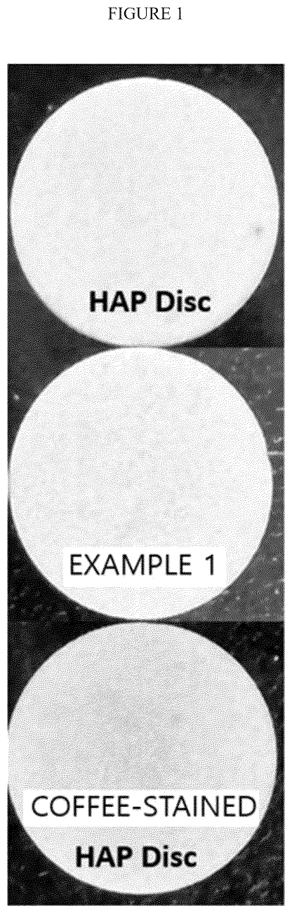

Reference Example 1 is an existing whitening toothpaste that is available on the market, in which the pH is 3.5 to 5.5, the hydrogen peroxide content is 0.75 wt % of the total weight of the whitening toothpaste, and the water content is 32 to 45 wt % of the total weight of the whitening toothpaste, where the prepared whitening toothpaste is a product that does not contain calcium phosphate and sodium metaphosphate. Specifically, an hydroxyapatite(HAP) disc made of tablets having an appropriate size by using a HAP powder was purchased and prepared, and a coffee-stained HAP disc, which had been obtained by treating the HAP disc in a dilute hydrochloric acid solution, subsequently washing the HAP disc completely, and then immersing the HAP disc in a coffee solution to artificially contaminate the HAP disc, was used as a test piece. A dilute hydrochloric acid solution was used to facilitate coffee staining. The prepared HAP disc and coffee-stained HAP disc can be confirmed in FIG. 1 below.

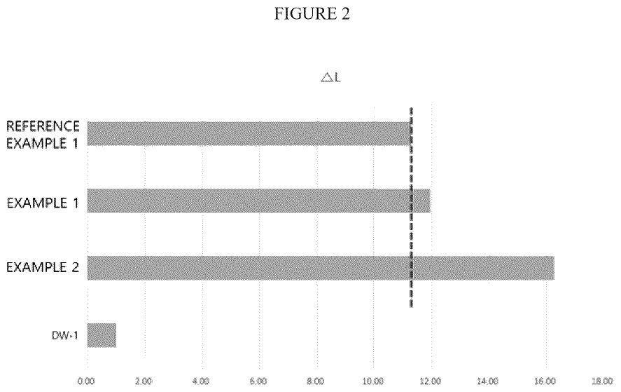

An initial L value (L indicates brightness, where a case where L is 100 is defined as pure white, and a case where L is 0 is defined as black) of the test piece of the coffee-stained HAP disc was measured with a color difference meter, and the oral composition for tooth whitening, which had been prepared as in the examples and comparative examples described above, was diluted in water, applied onto the test piece, and allowed to stand for 60 minutes under ordinary temperature conditions. Then, the test piece from which the composition for tooth whitening had been removed was washed and dried, and then the L value was measured again to calculate ΔL, the difference in L value before and after the treatment of the oral composition for tooth whitening. For each test piece, the color difference was measured five times before and after the treatment, the average value thereof was calculated, the color difference was calculated with the average value, and the results thereof are shown in Table 4 below and FIG. 2.

TABLE 4

| | Before treatment | After treatment | |
|---|---|---|---|
| | Average value | Average value | ΔL |
| Control (purified | 38.3 | 39.3 | 1.02 |
| water) | | | |
| Example 1 | 30.9 | 42.8 | 11.96 |
| Example 2 | 29.3 | 45.6 | 16.30 |
| Reference | 19.6 | 30.9 | 11.28 |
| Example 1 | | | |

With reference to Table 4 and FIG. 2, it can be confirmed that the oral composition for tooth whitening according to the present disclosure has an excellent whitening efficacy.

### Experimental Example 4: Evaluation of Safety of Oral Hard Tissue (In Vitro)

For each of the oral compositions for tooth whitening according to Examples 1 and the toothpaste of Reference Example 1, the safety of the oral hard tissue was evaluated.

Regarding the in vitro evaluation of the safety of the oral hard tissue, the in vitro evaluation of the safety of the oral hard tissue by using a test piece of a bovine tooth was carried out in accordance with the Chinese Evaluation Standard for Safety of Hard Tissue (GB/T 40002) according to a method in which the time for treating a test group was lengthened from 5 minutes to 30 minutes. Specifically, for the above-described evaluation, a process of subjecting two test pieces of bovine tooth to a treatment for 30 minutes with a sample diluent and then a treatment with an artificial saliva for 30 minutes was carried out under six cycle conditions. The initial microhardness of each test piece was measured four times, the average value thereof was calculated, and then the microhardness was measured four times after six cycles, and the average value thereof was calculated. The difference value therebetween is shown in Table 5 below. Herein, the microhardness was measured using a Vickers hardness meter.

TABLE 5

| | Bovine | Average value | Average value of | |
|---|---|---|---|---|
| | tooth test | of initial | microhardness after | Value of |
| | piece No. | microhardness | 6 cycles | difference |
| Reference | 1 | 329.25 | 288 | −41.25 |
| Example 1 | 2 | 321.25 | 302.25 | −19 |
| Example 1 | 1 | 340.75 | 345.5 | 4.75 |
| | 2 | 347.5 | 348.75 | 1.25 |

With reference to Table 5, it could be confirmed that the oral composition for tooth whitening according to the present disclosure hardly shows a decrease in hardness after six cycles, even though the treatment time has been extended to 30 minutes in the existing evaluation standards for safety of hard tissue.

Although the exemplary embodiments of the present disclosure have been described, a person skilled in the art to which the present disclosure belongs shall be able to understand that the present disclosure may be implemented in other specific forms without changing the technical ideas or essential features. Therefore, it should be understood that the examples described above should be understood as exemplary and should not be limited thereto.

1. An oral composition for tooth whitening, comprising:
a peroxide;
a polyvalent phosphate;
a divalent metal phosphate; and
water;
wherein the oral composition for tooth whitening has a pH of 5.5 or more.

2. The oral composition for tooth whitening according to claim 1,
wherein the peroxide is one or more selected from the group consisting of substances that are capable of releasing hydrogen peroxide, and
the substances that are capable of releasing hydrogen peroxide include hydrogen peroxide, carbamide peroxide, calcium peroxide, sodium percarbonate, and sodium perborate.

3. The oral composition for tooth whitening according to claim 1,
wherein the polyvalent phosphate is one or more selected from the group consisting of polyvalent phosphates that have a linear form, cyclic form, or branched form, and
the polyvalent phosphate having a linear form, cyclic form, or branched form includes sodium metaphosphate, sodium pyrophosphate, sodium polyphosphate, potassium pyrophosphate, acidic sodium pyrophosphate, acidic sodium metapolyphosphate, and acidic sodium polyphosphate.

4. The oral composition for tooth whitening according to claim 1,
wherein the divalent metal phosphate is one or more selected from the group consisting of calcium, magnesium, copper, and a phosphate thereof.

5. The oral composition for tooth whitening according to claim 1,
wherein, based on a total amount of the oral composition for tooth whitening, a content of the peroxide is 0.01 wt % to 10 wt %,
a content of the polyvalent phosphate is 0.01 wt % to 5 wt %,
a content of the divalent metal phosphate is 0.01 wt % to 2 wt %, and
a content of the water is 5 wt % to 55 wt %.

6. The oral composition for tooth whitening according to claim 1, further comprising:
one or more selected from the group consisting of a binding agent and a solubilizing agent.

7. The oral composition for tooth whitening according to claim 6,
wherein the binding agent is one or more selected from the group consisting of substances that are capable of forming a hydrogen bond with hydrogen peroxide, and
the substances that are capable of forming a hydrogen bond with the hydrogen peroxide include polyvinyl pyrrolidone, polyacrylamide, a polyvinyl pyrrolidone-vinyl acetate copolymer, and poly(2-ethyl-2-oxazoline).

8. The oral composition for tooth whitening according to claim 6,
wherein a weight ratio of the binding agent to the peroxide is more than 0.27.

9. The oral composition for tooth whitening according to claim 6,
wherein the solubilizing agent is a surfactant having an HLB of 14 to 23, and
the surfactant having an HLB of 14 to 23 is one or more selected from the group consisting of a poloxamer 407, a poloxamer 184, a polysorbate 60, a polysorbate 80, a polysorbate 20, a polyoxyethylene hydrogenated castor oil-40 (HCO 40), and a polyoxyethylene hydrogenated castor oil-60 (HCO 60).

10. The oral composition for tooth whitening according to claim 1, further comprising:
one or more additives selected from the group consisting of an abrasive agent, a viscosity adjusting agent, a pH adjusting agent, a wetting agent, a foaming agent, a sweetening agent, and a flavoring agent.
